(19) United States
(12) Patent Application Publication
(10) Pub. No.: US 2015/0250455 A1
(43) Pub. Date: Sep. 10, 2015

(54) MEDICAL IMAGE PROCESSING APPARATUS AND METHOD, AND COMPUTER-READABLE RECORDING MEDIUM

(71) Applicant: SAMSUNG ELECTRONICS CO., LTD., Suwon-si (KR)

(72) Inventors: Young-yoon LEE, Suwon-si (KR); Jong-geun PARK, Seoul (KR); Jin-woo YIM, Seongnam-si (KR); Ki-won SOHN, Seoul (KR); Ho-kyung KANG, Seoul (KR)

(73) Assignee: SAMSUNG ELECTRONICS CO., LTD., Suwon-si (KR)

(21) Appl. No.: 14/636,598

(22) Filed: Mar. 3, 2015

(30) Foreign Application Priority Data
Mar. 4, 2014 (KR) 10-2014-0025670

(51) Int. Cl. A61B 8/08; G06K 9/62

(52) U.S. Cl. CPC A61B 8/5215; G06K 9/6255; A61B 8/0808; A61B 8/483

(57) ABSTRACT

A medical image processing apparatus includes an image processor configured to detect an anatomical organ from a three-dimensional (3D) brain image and determine a plane-of-interest (POI) from the 3D brain image, based on the detected anatomical organ, and an output unit configured to output an image of the POI.

### CROSS-REFERENCE TO RELATED APPLICATION

This application claims priority from Korean Patent Application No. 10-2014-0025670, filed on Mar. 4, 2014, in the Korean Intellectual Property Office, the disclosure of which is incorporated herein in its entirety by reference.

### BACKGROUND

1. Field

Apparatuses and methods consistent with exemplary embodiments relate to a medical image processing apparatus and method, and a computer-readable recording medium.

2. Description of the Related Art

With the advance of technology for capturing a three-dimensional (3D) medical image, a 3D imaging technique is widely used for capturing a medical image, e.g., a brain image. For example, for the prediction of a developmental age and diagnosis of any deformation of the fetus, an ultrasound fetus measurement technology captures a two-dimensional (2D) ultrasound image or 3D ultrasound image of a fetus to diagnose brain development and brain deformation of the fetus. A user (for example, a doctor or an ultrasound technician) adjusts a position and a direction in which an ultrasound measurement is acquired, and manipulates obtained data, thereby locating a desired plane or image in a 3D ultrasound image. In the related art, a user needs to manually locate a desired plane or image.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments provide a medical image processing apparatus and method that may automatically determine a plane-of-interest (POI), as desired by a user, in a three-dimensional (3D) brain image.

One or more exemplary embodiments provide a medical image processing apparatus and method that may automatically measure parameters in a brain image.

Additional aspects will be set forth in part in the description which follows and, in part, will be apparent from the description, or may be learned by practice of the exemplary embodiments.

According to an aspect of an exemplary embodiment, a medical image processing apparatus includes: an image processor, e.g. an image processor, that detects an anatomical organ from a three-dimensional (3D) brain image, and determines a plane-of-interest (POI) from the 3D brain image, based on the detecting of the anatomical organ; and an output unit, e.g. a display, that outputs an image of the POI.

The POI may be a sagittal plane. In an operation of detecting the anatomical organ, the image processor may detect an elliptical shape of a maximum size while moving a two-dimensional (2D) plane in the 3D brain image, and detect a cavum septi pellucidi and a cerebellum, and in an operation of determining the POI, the image processor may determine the 2D plane, in which the elliptical shape of the maximum size is detected and the cavum septi pellucidi and a cerebellum are detected, as the sagittal plane.

The POI may be a transthalamic plane. In an operation of detecting the anatomical organ, the image processor may detect a cavum septi pellucidi from a sagittal plane of the 3D brain image, set at least one candidate plane of the transthalamic plane which is vertical to the sagittal plane based on the detected cavum septi pellucidi, and detect a skull and a trident shape from the at least one candidate plane of the transthalamic plane, and in an operation of determining the POI, the image processor may determine a candidate plane of the transthalamic plane, in which a size of the skull has a maximum value and the trident shape is detected, as the transthalamic plane.

In an operation of setting the at least one candidate plane of the transthalamic plane, the image processor may set, as candidate planes of the transthalamic plane, a cavum septi pellucidi plane which is vertical to the sagittal plane and includes a straight line parallel to a long direction of the cavum septi pellucidi and at least one plane parallel to the cavum septi pellucidi plane.

When a change aspect of the size of the skull deviates from a reference range, the image processor may re-detect the sagittal plane.

The image processor may measure the size of the skull in the transthalamic plane.

The POI may be a transventricular plane. In an operation of detecting the analogical organ, the image processor may detect a cavum septi pellucidi from a sagittal plane of the 3D brain image, set at least one candidate plane of the transventricular plane which is vertical to the sagittal plane, based on the detected cavum septi pellucidi, and detect a choroid plexus and a ventricle from the at least one candidate plane of the transventricular plane, and in an operation of determining the POI, the image processor may determine the transventricular plane among the at least one candidate plane of the transventricular plane according to the detection result of the choroid plexus and ventricle.

In an operation of setting the at least one candidate plane of the transthalamic plane, the image processor may set, as candidate planes of the transthalamic plane, a cavum septi pellucidi plane which is vertical to the sagittal plane and includes a straight line parallel to a long direction of the cavum septi pellucidi and at least one plane parallel to the cavum septi pellucidi plane.

In an operation of determining the transventricular plane, the image processor may determine the transventricular plane, based on at least one of a contrast of a boundary of a region corresponding to the choroid plexus, a size of the choroid plexus, and a contrast of a boundary of a region corresponding to the ventricle.

The image processor may detect a center of the choroid plexus region and a center of the ventricle region, determine a central line bisecting a line connecting the center of the choroid plexus region and the center of the ventricle region, determine a first straight line approximating an upper boundary of the choroid plexus region and the ventricle region and a second straight line approximating a lower boundary of the choroid plexus region and the ventricle region, and determine, as a ventricle size, a distance between two points at which the first and second straight lines intersect the central line.

In an operation of determining the first and second straight lines, the image processor may determine the first and second straight lines in consideration of an angle between the central line and the first straight line and an angle between the central line and the second straight line.

The POI may be a transcerebellar plane. In an operation of detecting the anatomical organ, the image processor may detect a cavum septi pellucidi and a cerebellum from a sagittal plane, set at least one candidate plane of the transcerebellar plane which includes a straight line connecting the cavum septi pellucidi and the cerebellum and is vertical to the sagittal plane, and detect the cerebellum from the at least one candidate plane of the transcerebellar plane, and in an operation of determining the POI, the image processor may determine the transcerebellar plane according to a result of the cerebellum detected from the at least one candidate plane of the transcerebellar plane.

In an operation of detecting the cavum septi pellucidi and the cerebellum from the sagittal plane, the image processor may detect a skull from the sagittal plane, find a symmetrical line of the skull, and detect two 8-shaped circles or ellipses vertically contacting the symmetrical line to detect the cerebellum.

The image processor may measure a length of a line, which connects both vertical-direction ends of the two 8-shaped circles or ellipses in the transcerebellar plane, to measure a size of the cerebellum.

The image processor may detect a cistern magna from the transcerebellar plane, and measure a distance between a point, at which the two circles or ellipses of the cerebellum contact each other, and the cistern magna to measure a spinal fluid space.

When at least one of a brightness difference, shape difference, and size difference between the two circles or ellipses of the cerebellum is equal to or greater than a reference range, the image processor may re-detect the sagittal plane.

According to an aspect of an exemplary embodiment, a medical image processing method includes: detecting an anatomical organ from a three-dimensional (3D) brain image; determining a plane-of-interest (POI) from the 3D brain image, based on the detecting of the anatomical organ; and outputting an image of the POI.

The POI may be a sagittal plane, the detecting of the anatomical organ may include: detecting an elliptical shape of a maximum size while moving a two-dimensional (2D) plane in the 3D brain image; and detecting a cavum septi pellucidi and a cerebellum, and the determining of the POI may include determining the 2D plane, in which the elliptical shape of the maximum size is detected and the cavum septi pellucidi and a cerebellum are detected, as the sagittal plane.

The POI may be a transthalamic plane, the detecting of the anatomical organ may include: detecting a cavum septi pellucidi from a sagittal plane of the 3D brain image; setting at least one candidate plane of the transthalamic plane which is vertical to the sagittal plane based on the detected cavum septi pellucidi; and detecting a skull and a trident shape from the at least one candidate plane of the transthalamic plane, and the determining of the POI may include determining a candidate plane of the transthalamic plane, in which a size of the skull has a maximum value and the trident shape is detected, as the transthalamic plane.

The setting of the at least one candidate plane of the transthalamic plane may include setting, as candidate planes of the transthalamic plane, a cavum septi pellucidi plane which is vertical to the sagittal plane and includes a straight line parallel to a long direction of the cavum septi pellucidi and at least one plane parallel to the cavum septi pellucidi plane.

The medical image processing method may further include, when a change aspect of the size of the skull deviates from a reference range, re-detecting the sagittal plane.

The medical image processing method may further include measuring the size of the skull in the transthalamic plane.

The POI may be a transventricular plane, the detecting of the anatomical organ may include: detecting a cavum septi pellucidi from a sagittal plane of the 3D brain image; setting at least one candidate plane of the transventricular plane which is vertical to the sagittal plane, based on the detected cavum septi pellucidi; and detecting a choroid plexus and a ventricle from the at least one candidate plane of the transventricular plane, and the determining of the POI may include determining the transventricular plane among the at least one candidate plane of the transventricular plane according to the detection result of the choroid plexus and ventricle.

The setting of the at least one candidate plane of the transventricular plane may include setting, as candidate planes of the transthalamic plane, a cavum septi pellucidi plane which is vertical to the sagittal plane and includes a straight line parallel to a long direction of the cavum septi pellucidi and at least one plane parallel to the cavum septi pellucidi plane.

The determining of the transventricular plane may include determining the transventricular plane, based on at least one of a contrast of a boundary of a region corresponding to the choroid plexus, a size of the choroid plexus, and a contrast of a boundary of a region corresponding to the ventricle.

The medical image processing method may further include: detecting a center of the choroid plexus region and a center of the ventricle region; determining a central line bisecting a line connecting the center of the choroid plexus region and the center of the ventricle region; determining a first straight line approximating an upper boundary of the choroid plexus region and the ventricle region and a second straight line approximating a lower boundary of the choroid plexus region and the ventricle region; and determining, as a ventricle size, a distance between two points at which the first and second straight lines intersect the central line.

The determining of the distance as the ventricle size may include determining the first and second straight lines in consideration of an angle between the central line and the first straight line and an angle between the central line and the second straight line.

The POI may be a transcerebellar plane, the detecting of the anatomical organ may include: detecting a cavum septi pellucidi and a cerebellum from a sagittal plane; setting at least one candidate plane of the transcerebellar plane which includes a straight line connecting the cavum septi pellucidi and the cerebellum and is vertical to the sagittal plane; and detecting the cerebellum from the at least one candidate plane of the transcerebellar plane, and the determining of the POI may include determining the transcerebellar plane according to a result of the cerebellum detected from the at least one candidate plane of the transcerebellar plane.

The detecting of the cavum septi pellucidi and the cerebellum may include: detecting a skull from the sagittal plane, finds a symmetrical line of the skull; and detecting two 8-shaped circles or ellipses vertically contacting the symmetrical line to detect the cerebellum.

The medical image processing method may further include measuring a length of a line, which connects both vertical-direction ends of the two 8-shaped circles or ellipses in the transcerebellar plane, to measure a size of the cerebellum.

The medical image processing method may further include: detecting a cistern magna from the transcerebellar plane; and measuring a distance between a point, at which the two circles or ellipses of the cerebellum contact each other, and the cistern magna to measure a spinal fluid space.

The medical image processing method may further include re-detecting the sagittal plane when at least one of a brightness difference, shape difference, and size difference between the two circles or ellipses of the cerebellum is equal to or greater than a reference range.

According to an exemplary embodiment, provided is a non-transitory computer-readable storage medium storing a program which, when executed by a computer, performs a medical image processing method including: detecting an anatomical organ from a three-dimensional (3D) brain image; determining a plane-of-interest (POI) from the 3D brain image, based on the detection result of the anatomical organ; and outputting an image of the POI.

### BRIEF DESCRIPTION OF THE DRAWINGS

The above and/or other aspects will become more apparent by describing certain exemplary embodiments with reference to the accompanying drawings, in which:

FIG. 1 is a diagram illustrating a structure of a medical image processing apparatus according to an exemplary embodiment;

FIG. 2 is a flowchart illustrating a medical image processing method according to an exemplary embodiment;

FIG. 3 is a diagram for describing an operation of detecting a sagittal plane of a brain, according to an exemplary embodiment;

FIG. 4 is a flowchart illustrating an operation of detecting a sagittal plane of a brain, according to an exemplary embodiment;

FIG. 5 is a diagram illustrating a plane of a skull in a sagittal plane;

FIG. 6 is a diagram for describing an operation of detecting a skull, according to an exemplary embodiment;

FIG. 7 is a diagram illustrating an image on a midsagittal plane from which a cavum septi pellucidi (CSP) and a cerebellum are detected, according to an exemplary embodiment;

FIG. 8 is a diagram illustrating an example of a POI in a sagittal plane, according to an exemplary embodiment;

FIG. 9 is a flowchart illustrating an operation of detecting a transthalamic plane, according to an exemplary embodiment;

FIG. 10 is a diagram for describing an operation of setting a candidate plane of a transthalamic plane, according to an exemplary embodiment;

FIG. 11 is a diagram illustrating a candidate transthalamic plane according to an exemplary embodiment;

FIG. 12 is a diagram for describing an operation of measuring a size of a skull in a transthalamic plane, according to an exemplary embodiment;

FIG. 13 is a diagram for describing an operation of detecting a skull region, according to an exemplary embodiment;

FIG. 14 is a flowchart illustrating an operation of determining a transventricular plane, according to an exemplary embodiment;

FIG. 15 is a diagram illustrating a transventricular plane according to an exemplary embodiment;

FIG. 16 is a diagram illustrating an example of a template used in a template matching technique according to an exemplary embodiment;

FIG. 17 is a diagram illustrating an operation of measuring a ventricle size, according to an exemplary embodiment;

FIG. 18 is a diagram illustrating a choroid plexus (CP) region and a ventricle region according to an exemplary embodiment;

FIG. 19 is a flowchart illustrating an operation of determining a transcerebellar plane, according to an exemplary embodiment;

FIG. 20 is a diagram for describing an operation of determining a transcerebellar plane, according to an exemplary embodiment;

FIG. 21 is a diagram illustrating a candidate transcerebellar plane according to an exemplary embodiment;

FIG. 22 is a diagram illustrating a cerebellum region according to an exemplary embodiment;

FIG. 23 is a flowchart illustrating an operation of measuring a spinal fluid space, according to an exemplary embodiment;

FIG. 24 is a diagram for describing a spinal fluid space according to an exemplary embodiment;

FIG. 25 is a flowchart illustrating an operation of detecting a POI, according to an exemplary embodiment; and

FIG. 26 is a block diagram illustrating a configuration of an ultrasound diagnostic apparatus according to an exemplary embodiment.

### DETAILED DESCRIPTION

Certain exemplary embodiments are described in detail below with reference to the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the figures, to explain aspects of the disclosure. Expressions such as “at least one of,” when preceding a list of elements, modify the entire list of elements and do not modify the individual elements of the list.

In this disclosure below, when it is described that one comprises (or includes or has) some elements, it should be understood that it may comprise (or include or has) only those elements, or it may comprise (or include or have) other elements as well as those elements if there is no specific limitation. Moreover, each of terms such as “ . . . unit”, “ . . . apparatus” and “module” described in the specification denotes an element for performing at least one function or operation, and may be implemented in hardware, software or the combination of hardware and software.

The term “ultrasonic image” used herein denotes an image of an object acquired by using an ultrasonic wave. Also, the term “object” used herein may include a person, an animal, a part of the person, or a part of the animal. For example, an object may include an organ such as a liver, a heart, a womb, a brain, breasts, an abdomen, or the like, or a blood vessel. Also, the term “object” may include a phantom. A phantom denotes a material having a volume that is very close to a density and an effective atomic number of an organism, and may include a spherical phantom having a characteristic similar to a physical body.

Moreover, the term “user” used herein is a medical expert, and may be a doctor, a nurse, a medical technologist, a medical image expert, or the like, or may be an engineer who repairs a medical apparatus. However, the user is not limited thereto.

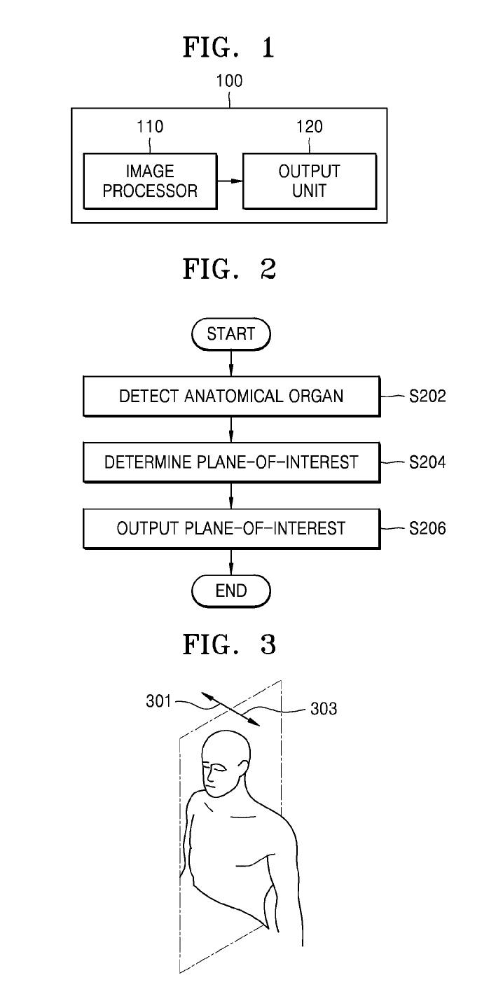

FIG. 1 is a diagram illustrating a structure of a medical image processing apparatus 100 according to an exemplary embodiment. The medical image processing apparatus 100 according to an exemplary embodiment includes an image processor 110, and an output unit 120. For example, the output unit 120 may include a display.

The image processor 110 processes an input medical image. The image processor 110 may detect an anatomical organ from a three-dimensional (3D) brain image, and determine a plane of interest (POI) from the 3D brain image based on the detection of the anatomical organ.

Examples of the 3D brain image may include a 3D ultrasound image, a 3D computed tomography (CT) image, and a 3D magnetic resonance (MR) image. Also, the 3D brain image may be an image which is captured in real-time or an image which is captured and stored.

Examples of the POI may include a sagittal plane, a transthalamic plane, a transventricular plane, and a transcerebellar plane.

Examples of the anatomical organ may include a skull, a cavum septi pellucidi (CSP), a choroid plexus (CP), a cerebellum, a spinal fluid, and a ventricle.

When detecting the anatomical organ, the image processor 110 may use, for example, an adaptive thresholding technique, a region growing technique, an elliptical approximation technique, or a template matching technique. A medical image such as an ultrasound image may have limited sharpness, and thus, by using the above techniques, it is possible to more accurately detect the anatomical organ.

When determining the POI from the 3D brain image, the image processor 110 may detect the POI based on the detection of the anatomical organ. For example, the image processor 110 may detect the POI based on whether the anatomical organ is included in a corresponding plane, whether a size of the anatomical organ has the maximum value in the corresponding plane, and whether the anatomical organ is clearly shown in the corresponding plane.

The image processor 110 may search for a two-dimensional (2D) plane, from which the anatomical organ is detected, and detect the POI while moving the 2D plane in the 3D brain image. For example, the image processor 110 may capture a 3D brain image in real time, and detect a POI while moving a 2D plane. As another example, the image processor 110 may detect a POI while moving a 2D plane in a stored 3D brain image.

The image processor 110 may store the detected POI in a storage unit automatically or according to a user input.

The output unit 120 outputs the detected POI.

According to an exemplary embodiment, the output unit 120 may include a display that displays the POI.

According to another exemplary embodiment, the output unit 120 may output an image file of the POI. In this case, the image file of the POI may be stored in the storage unit, or transmitted to another electronic device.

FIG. 2 is a flowchart illustrating a medical image processing method according to an exemplary embodiment.

In operation 5202, the medical image processing method detects an anatomical organ from a 3D brain image.

When the anatomical organ is detected, the medical image processing method determines a POI from the 3D brain image based on the detection of the anatomical organ, in operation 5204. Examples of the POI include a sagittal plane, a transthalamic plane, a transventricular plane, and a transcerebellar plane. Examples of the anatomical organ may include a skull, a cavum septi pellucidi (CSP), a choroid plexus (CP), a cerebellum, a spinal fluid, and a ventricle.

Subsequently, in operation 5206, the medical image processing method outputs the POI. For example, the POI may be displayed, stored as an image file in the storage unit, or transmitted to another electronic device.

FIG. 3 is a diagram for describing an operation of detecting a sagittal plane of a brain, according to an exemplary embodiment.

The sagittal plane of the brain denotes a plane in which a brain is vertically cut in a direction from a front portion to a back portion of the brain with respect to a central line of the brain. The image processor 110 may detect a skull, a CSP, and a cerebellum while moving a 2D plane in which the brain is vertically cut in the direction from the front portion to the back portion of the brain, for detecting a sagittal plane. The 2D plane may move in a left direction 301 or a right direction 303 with respect to the central line of the brain, as shown in FIG. 3.

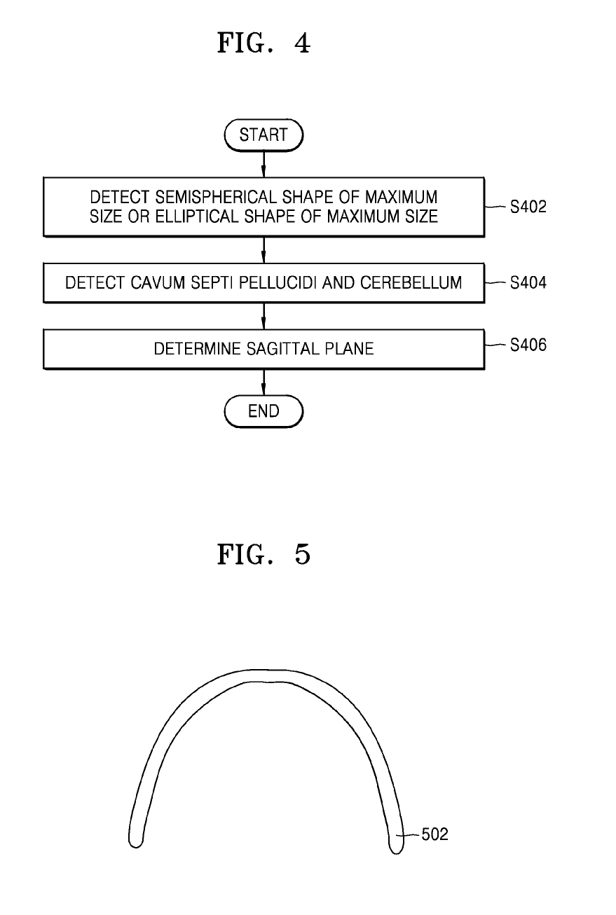

FIG. 4 is a flowchart illustrating an operation of detecting a sagittal plane of a brain, according to an exemplary embodiment.

In operation S402, the image processor 110 detects a semispherical shape of the maximum size or an elliptical shape of the maximum size while moving a 2D plane in which a brain is vertically cut in the direction from the front portion to the back portion of the brain, for detecting a sagittal plane. In this case, using the adaptive thresholding technique, the image processor 110 may detect a region having a brightness value greater than an ambient brightness value, and determine a candidate region of a skull. The adaptive thresholding technique is a technique that dynamically changes a reference value when binarizing a pixel value of an image.

The image processor 110 detects a semispherical shape from the candidate region of the skull by using a regression analysis technique, and detects an elliptical shape which is generated by the semispherical shape. Also, the image processor 110 detects the greatest elliptical shape by using the regression analysis technique.

FIG. 5 is a diagram illustrating a plane of a skull in a sagittal plane.

As illustrated in FIG. 5, a skull 502 may have a semispherical shape. The image processor 110 may detect the semispherical shape from a 2D plane in which a brain is vertically cut in a direction from a front portion to a back portion of the brain in a 3D brain image, thereby detecting the skull 502.

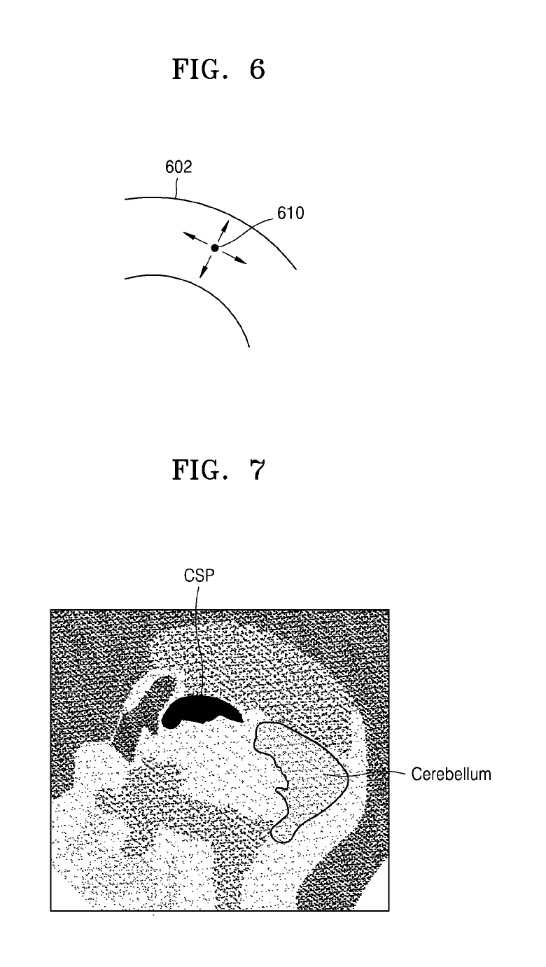

FIG. 6 is a diagram for describing an operation of detecting a skull, according to an exemplary embodiment.

According to an exemplary embodiment, when detecting the skull, a region growing technique may be used. For example, as illustrated in FIG. 6, a seed 610 may be set in a region having a brightness value, and a skull region 602 may be determined by using the region growing technique with respect to the seed 610.

Referring again to FIG. 4, in operation S404, the image processor 110 detects a CSP and a cerebellum from the 2D plane.

FIG. 7 is a diagram illustrating an image on a midsagittal plane from which a CSP and a cerebellum are detected, according to an exemplary embodiment.

As illustrated in FIG. 7, a sagittal plane includes a CSP and a cerebellum. According to an exemplary embodiment, the image processor 110 may detect a shape of the CSP and a shape of the cerebellum from the 2D plane by using the template matching technique.

Referring again to FIG. 4, in operation 5406, the image processor 110 determines, as a sagittal plane, a 2D plane in which the skull has the maximum size and the CSP and the cerebellum are all detected.

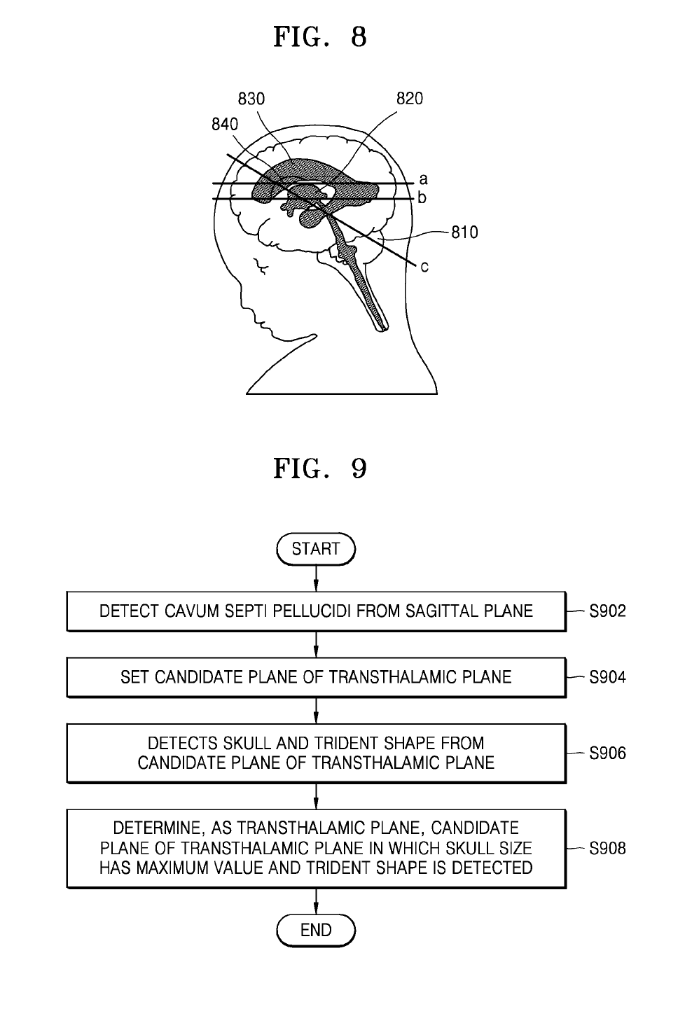

FIG. 8 is a diagram illustrating an example of a POI in a sagittal plane, according to an exemplary embodiment. FIG. 8 illustrates a shape in which a structure such as a lateral ventricle is projected on a 2D plane.

According to an exemplary embodiment, the POI may be at least one of a transthalamic plane which cuts the brain along a line b, a transventricular plane which cuts the brain along a line a, and a transcerebellar plane which cuts the brain alone a line c. In FIG. 8, a reference numeral “810” denotes a cerebellum, a reference numeral “820” refers to a third ventricle, and a reference numeral “830” denotes a lateral ventricle, which is projected on a sagittal plane. The transthalamic plane denotes a 2D plane which is vertical to the sagittal plane and is cut to include a CSP 840. The transventricular plane denotes a plane which is vertical to the sagittal plane, contacts a lower portion of the CSP 840, and is parallel to the transthalamic plane and in which a CP and a ventricle are detected. The transcerebellar plane denotes a plane which is vertical to the sagittal plane and passes through the cerebellum 810.

FIG. 9 is a flowchart illustrating an operation of detecting a transthalamic plane, according to an exemplary embodiment.

In operation S902, the image processor 110 detects a CSP from a sagittal plane. The CSP, as illustrated in FIG. 7, may be detected from the sagittal plane.

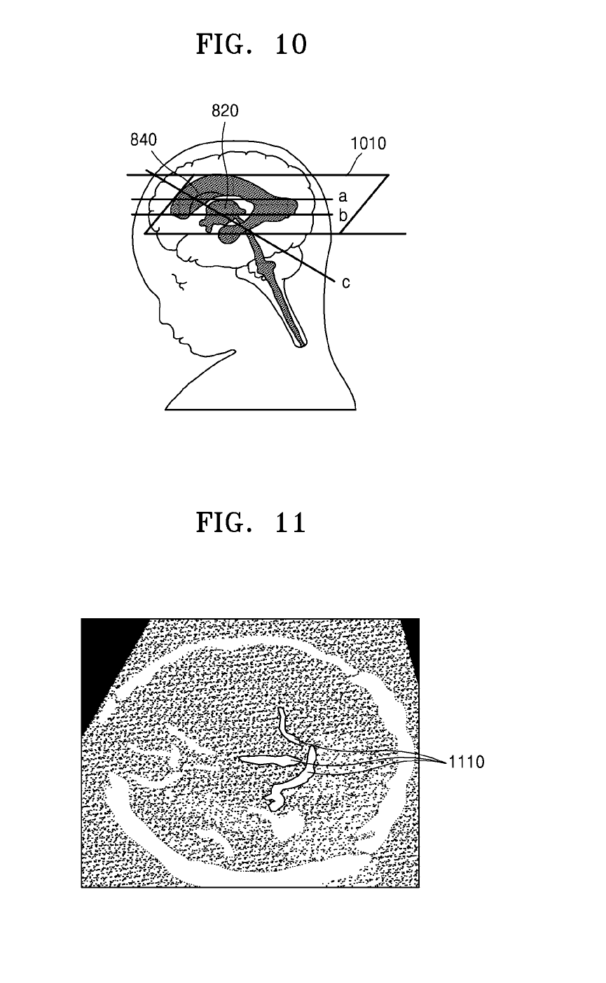

FIG. 10 is a diagram for describing an operation of setting a candidate plane for a transthalamic plane (i.e., a plane which is a candidate for the transthalamic plane), according to an exemplary embodiment.

Referring to FIGS. 9 and 10, in operation S904, the image processor 110 sets a plane 1010 (hereinafter “CSP plane”), which is vertical to a sagittal plane and contacts a lower end of the CSP 840, as a candidate plane of a transthalamic plane. In this case, the image processor 110 may set, as candidate planes of the transthalamic plane, a CSP plane (including a straight line, e.g., line b, contacting the lower end of the CSP 840) and at least one plane parallel to the CSP plane.

FIG. 11 is a diagram illustrating a candidate transthalamic plane according to an exemplary embodiment.

Referring to FIGS. 9 and 11, in operation S906, the image processor 110 detects a skull and regions 1110 forming a trident shape from the candidate plane of the transthalamic plane while moving the candidate plane of the transthalamic plane.

The skull has an elliptical shape, having a brightness value greater than an ambient brightness value, and two elliptical curves including an upper elliptical curve and a lower elliptical curve. In this case, the upper elliptical curve and the lower elliptical curve of the skull may be detected by using the region growing technique.

The regions 1110 forming a trident shape denotes a white portion having a trident shape which is generated by a thalami and a hyppocampal gyrus.

In operation S908, the image processor 110 determines, as the transthalamic plane, the candidate plane of the transthalamic plane in which a skull size has the maximum value and the regions 1110 forming a trident shape is detected.

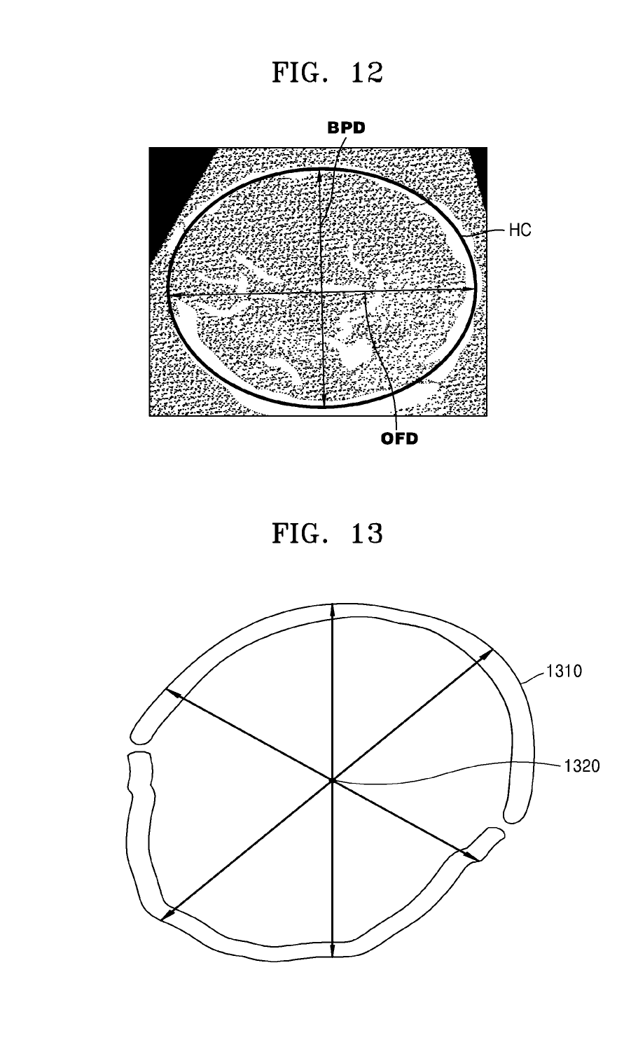

FIG. 12 is a diagram for describing an operation of measuring a size of a skull in a transthalamic plane, according to an exemplary embodiment.

According to an exemplary embodiment, a size of a skull may be detected from a transthalamic plane. FIG. 12 illustrates a transthalamic plane according to an exemplary embodiment. In FIG. 12, “HC” refers to a head circumference, “BPD” refers to a biparietal diameter of a skull, and “OFD” refers to an occipital-frontal diameter of the skull. The image processor 110 may detect an upper skull and a lower skull by using the elliptical approximation technique, for measuring the size of the skull. The head circumference HC may be defined as a circumferential length of an ellipse circumscribing the skull. The biparietal diameter BPD may be defined as a distance between a circumscribed point of the upper skull and an inscribed point of the lower skull. The occipital-frontal diameter OFD may be defined as an occipital-frontal diameter of the ellipse circumscribing the skull.

FIG. 13 is a diagram for describing an operation of detecting a skull region, according to an exemplary embodiment.

According to an exemplary embodiment, in order to increase an accuracy in measuring a skull size, a thicknesses according to a direction may be predicted from a center 1320 of the skull 1310, and an ellipse inscribing or circumscribing the skull 1310 may be approximated. For example, as illustrated in FIG. 13, by using a binarization technique or the adaptive thresholding technique, the image processor 110 may perform a skull region detecting operation in various directions from the center 1320 of the skull 1310, thereby accurately detecting a skull region. In this manner, a skull thickness may be more accurately predicted in each region of the skull 1310, and an ellipse inscribing the skull 1310 and an ellipse circumscribing the skull 1310 may be more accurately predicted.

The image processor 110 compares measurement values of the size of the skull 1310 detected from the candidate planes of the transthalamic plane to determine whether a difference in the measurement values deviates from a reference range, and when the difference in the measurement values does not deviate from the reference range, the image processor 110 determines, as the transthalamic plane, a candidate plane of the transthalamic plane in which a skull size has the maximum value. When the difference in the measurement value deviates from the reference range, the image processor 110 may again search for another sagittal plane, and determine the transthalamic plane based on the another sagittal plane.

A configuration for measuring the size of the skull 1310 may be applied to a 2D brain image. For example, as described above, the image processor 110 may measure the size of the skull 1310 in the 2D brain image corresponding to the transthalamic plane.

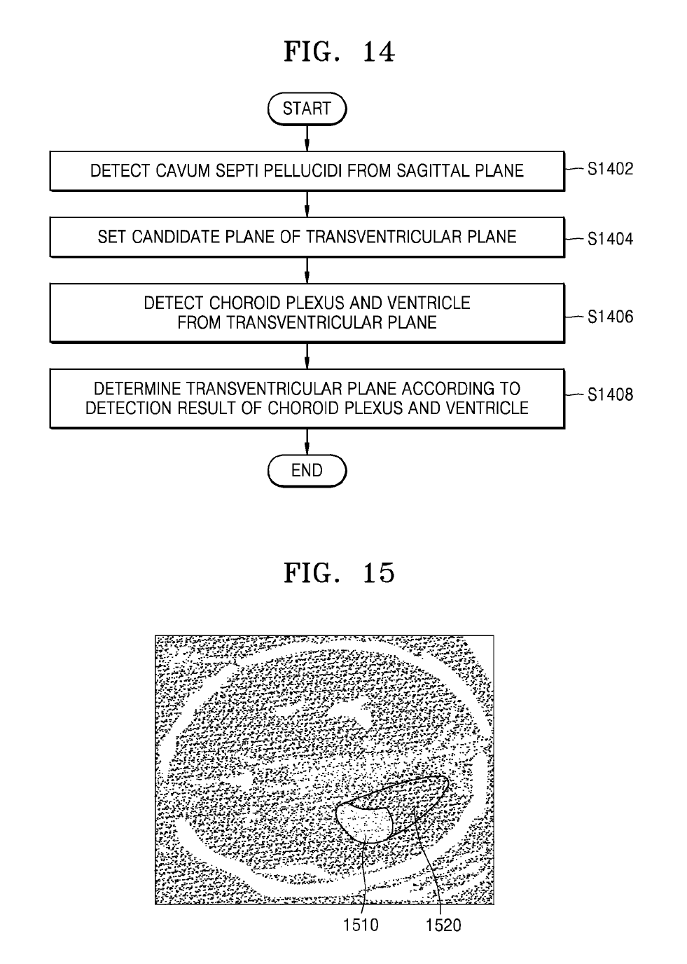

FIG. 14 is a flowchart illustrating an operation of determining a transventricular plane, according to an exemplary embodiment;

In operation S1402, the image processor 110 detects a CSP from a sagittal plane.

Subsequently, in operation S1404, the image processor 110 sets a candidate plane of a transventricular plane vertical to the sagittal plane based on the CSP. The candidate plane of the transventricular plane may include a CSP plane, which is vertical to a sagittal plane and includes a straight line contacting the CSP and parallel to an elongated direction of the CSP, and at least one plane parallel to the CSP plane.

Subsequently, in operation S1406, the image processor 110 detects a CP and a ventricle while moving the candidate plane of the transventricular plane.

FIG. 15 is a diagram illustrating a transventricular plane according to an exemplary embodiment.

The transventricular plane includes a CP region 1510 and a ventricle region 1520. The CP region 1510 is brighter than surrounding regions, and the ventricle region 1520 is darker than the surrounding regions. The image processor 110 may use the template matching technique, for detecting the CP region 1510 and the ventricle region 1520.

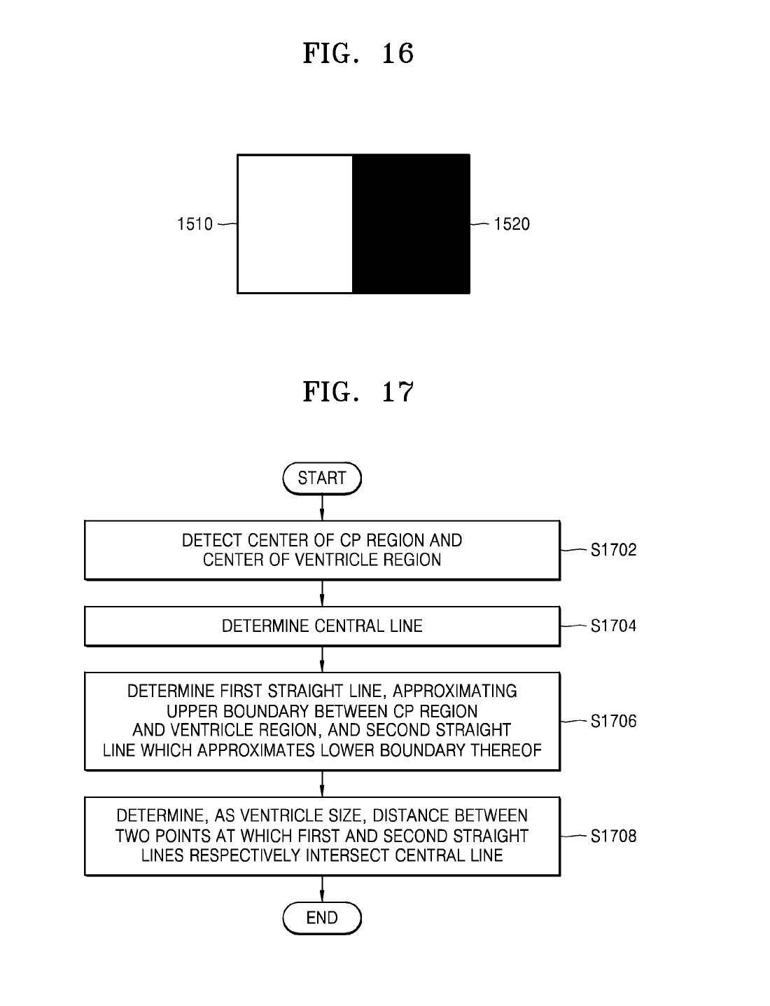

FIG. 16 is a diagram illustrating an example of a template used in the template matching technique according to an exemplary embodiment.

According to an exemplary embodiment, the CP region 1510 and the ventricle region 1520 are disposed adjacent to each other, and, in consideration that the CP region 1510 is brighter than the ventricle region 1520, as illustrated in FIG. 16, the CP region 1510 and the ventricle region 1520 may be detected by using the template matching technique, which uses a template in which a brighter region and a darker region are disposed adjacent to each other.

Referring again to FIG. 14, in operation S1408, the image processor 110 determines the transventricular plane from among candidate planes of the transventricular plane according to a result of detecting the CP and the ventricle. According to an exemplary embodiment, the image processor 110 may determine the transventricular plane based on at least one of a contrast of a boundary of the CP region 1510, a size of the CP, and a contrast of a boundary of the ventricle region 1520. For example, the image processor 110 determines, as the transventricular plane, a candidate plane of the transventricular plane in which the CP and the ventricle are clearly shown and each of the CP region 1510 and ventricle region 1520 has the maximum size.

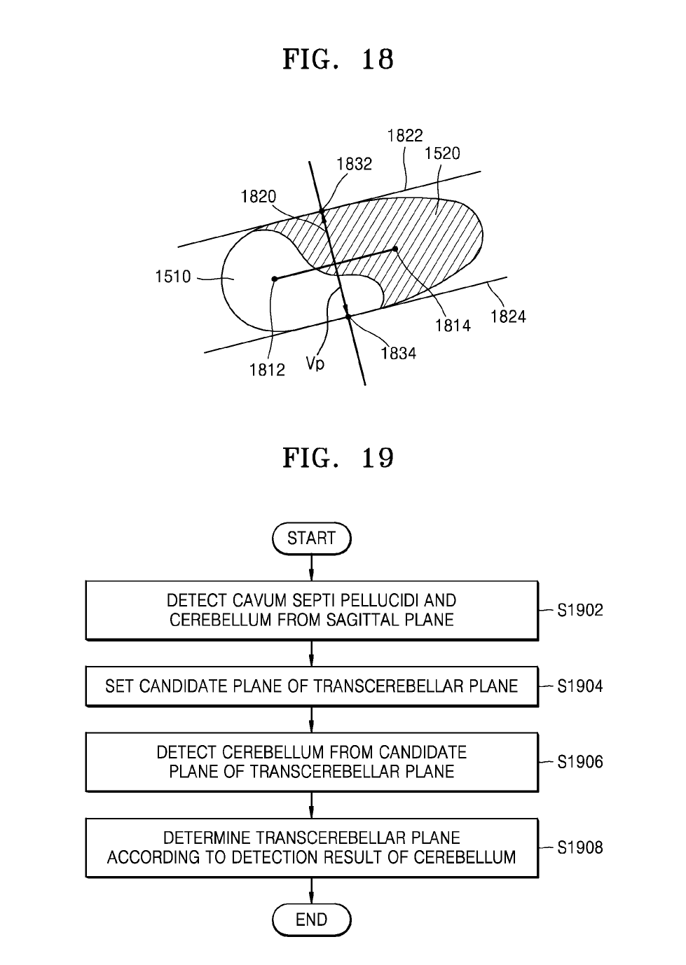

FIG. 17 is a diagram illustrating an operation of measuring a ventricle size, according to an exemplary embodiment. FIG. 18 is a diagram illustrating the CP region 1510 and the ventricle region 1520 according to an exemplary embodiment.

In operation S1702, when a transventricular plane is determined, the image processor 110 detects a center 1812 of the CP region 1510 and a center 1814 of the ventricle region 1520 from the transventricular plane.

According to an exemplary embodiment, the image processor 110 defines the CP region 1510 and the ventricle region 1520 by using the template matching technique. The image processor 110 detects a position, in which a value obtained by subtracting an average brightness value of brightness values of pixels corresponding to a black region of the template from an average brightness value of brightness values of pixels corresponding to a white region of the template is the maximum, from an image on the transventricular plane while moving the template of FIG. 16, and defines edges of the CP region 1510 and the ventricle region 1520 based on the detected position. Also, the image processor 110 binarizes the image on the transventricular plane based on an intermediate value between the average brightness value of the white region of the template and the average brightness value of the black region of the template, and defines the CP region 1510 and the ventricle region 1520. When the CP region 1510 and the ventricle region 1520 are defined, the image processor 110 calculates a centroid of the CP region 1510, and defines the centroid as the center 1812 of the CP region 1510. Also, the image processor 110 calculates a centroid of the ventricle region 1520, and defines the centroid as the center 1814 of the ventricle region 1520.

Subsequently, in operation S1704, the image processor 110 determines a central line 1820 between the center 1812 of the CP region 1510 and the center 1814 of the ventricle region 1520. For example, the central line 1820 may be determined as a straight line which is vertical to a line connecting the center 1812 of the CP region 1510 and the center 1814 of the ventricle region 1520 and passes through the center of the line.

Subsequently, in operation S1706, the image processor 110 determines a first straight line 1822 which approximates an upper boundary of the CP region 1510 and the ventricle region 1520, and a second straight line 1824 which approximates a lower boundary of the CP region 1510 and the ventricle region 1520. For example, the first and second straight lines 1822 and 1824 may be determined by using a principal component analysis technique or a linear regression analysis technique.

When determining the first straight line 1822, the image processor 110 may determine the first straight line 1822 so that an angle between the central line 1820 and the first straight line 1822 is within a reference range. Also, when determining the second straight line 1824, the image processor 110 may determine the second straight line 1824 so that the angle between the central line 1820 and the second straight line 1824 is within the reference range.

Subsequently, in operation S1708, the image processor 110 determines, as a ventricle size, a distance Vp between a point 1832 at which the first straight line 1822 intersects the central line 1820 and a point 1834 at which the second straight line 1824 intersects the central line 1820.

A configuration for measuring the ventricle size may be applied to a 2D brain image. For example, as described above, the image processor 110 may detect the CP region 1510 and the ventricle region 1520 to measure the ventricle size in a 2D image on the transthalamic plane.

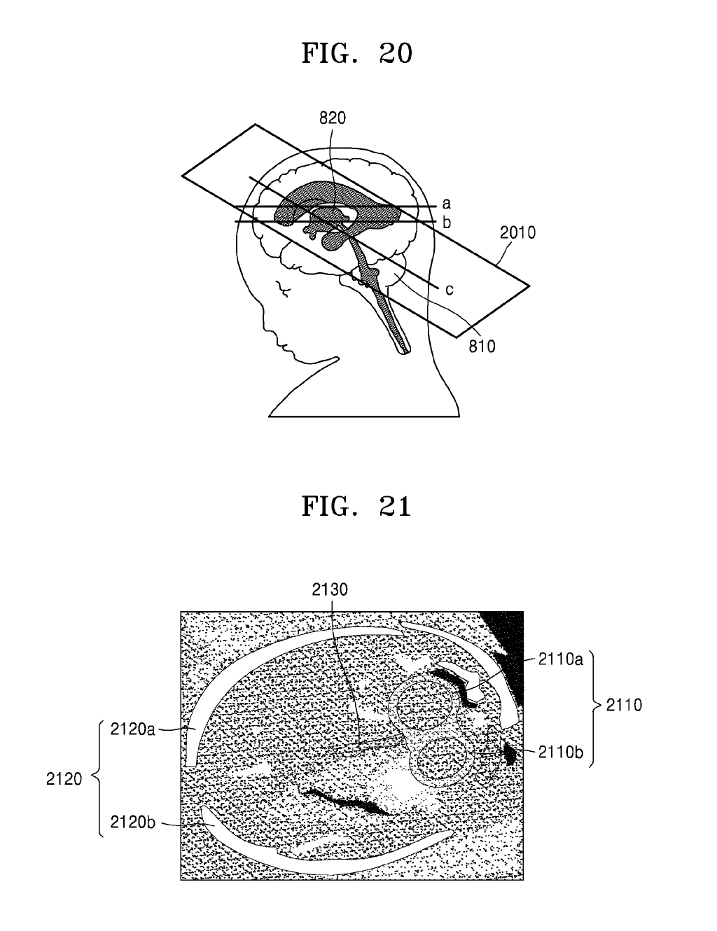

FIG. 19 is a flowchart illustrating an operation of determining a transcerebellar plane, according to an exemplary embodiment. FIG. 20 is a diagram for describing an operation of determining a transcerebellar plane, according to an exemplary embodiment.

In operation S1902, the image processor 110 detects a CSP 820 and a cerebellum 810 from a sagittal plane. For example, the image processor 110 may detect the CSP 820 and the cerebellum 810 by using the template matching technique.

Subsequently, in operation S1904, the image processor 110 sets at least one candidate plane 2010 of a transcerebellar plane which includes a straight line connecting the CSP 820 and the cerebellum 810 and is vertical to the sagittal plane.

FIG. 21 is a diagram illustrating a candidate transcerebellar plane according to an exemplary embodiment.

Referring to FIGS. 19 and 21, in operation S1906, the image processor 110 detects a cerebellum region 2110 having a shape similar to a figure “8” from a candidate plane of the transcerebellar plane. In this case, the image processor 110 may detect a symmetrical line region 2130 of a skull indicating a midsagittal plane, and approximate the cerebellum region 2110 to an upper elliptical shape 2110a and a lower elliptical shape 2110b. Also, the image processor 110 may detect a skull region 2120 from the candidate plane of the transcerebellar plane. The skull region 2120 may include an upper skull region 2120a and a lower skull region 2120b.

In operation S1908, the image processor 110 determines a transcerebellar plane according to a result of detecting the cerebellum on the candidate plane of the transcerebellar plane.

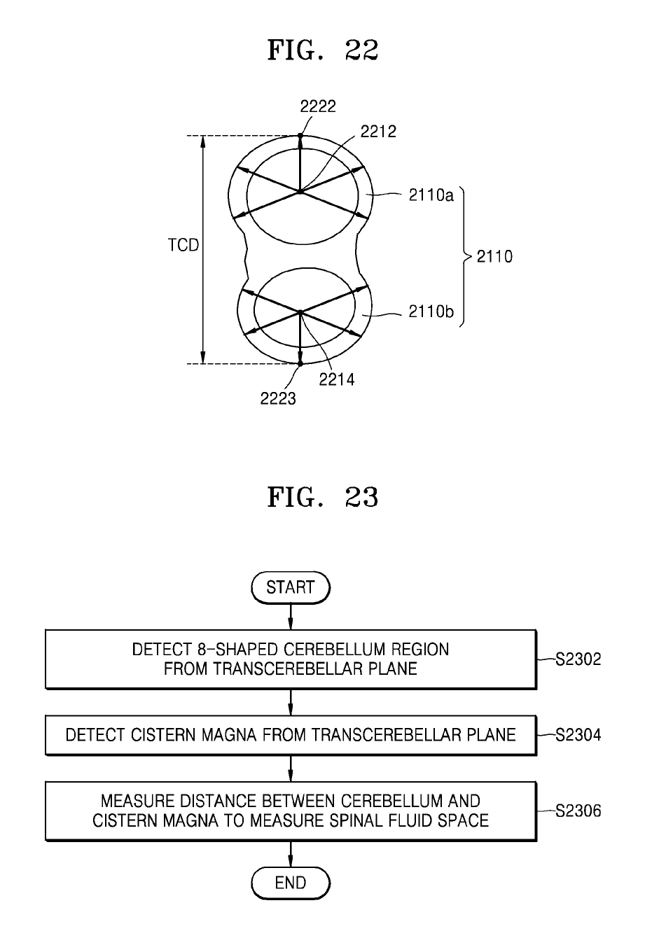

FIG. 22 is a diagram illustrating the cerebellum region 2110 according to an exemplary embodiment.

According to an exemplary embodiment, a size of a cerebellum may be determined from the cerebellum region 2110. As illustrated in FIG. 22, by using the binarizing technique or the adaptive thresholding technique, the image processor 110 may perform a cerebellum region determining operation in various directions from respective centers 2212 and 2214 of the upper elliptical shape 2110a and lower elliptical shape 2110b of the cerebellum region 2110, thereby accurately determining the cerebellum region 2110.

Moreover, the image processor 110 approximates an ellipse circumscribing each of the upper elliptical shape 2110a and lower elliptical shape 2110b of the cerebellum region 2110, and determines a highest point 2222 and a lowest point 2223 of the cerebellum region 2110 from the approximated circumscribed ellipse. The image processor 110 measures a distance (i.e., transcerebellar diameter (TCD)) between the highest point 2222 and the lowest point 2223 to determine a cerebellum size.

A configuration for measuring the cerebellum size may be applied to a 2D brain image. For example, as described above, the image processor 110 may detect the cerebellum region 2110 from an image on the transcerebellar plane to measure the cerebellum size.

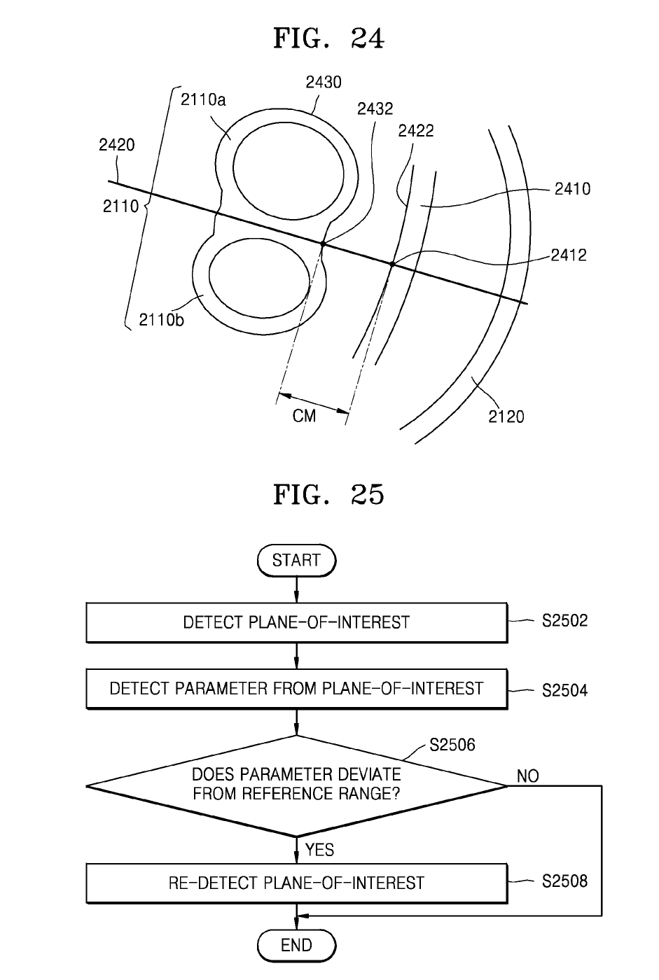

FIG. 23 is a flowchart illustrating an operation of measuring a spinal fluid space, according to an exemplary embodiment. FIG. 24 is a diagram for describing a spinal fluid space according to an exemplary embodiment.

In operation S2302, the image processor 110 detects an “8”-shaped cerebellum region 2110 from an image on a transcerebellar plane. In operation S2304, the image processor detects a boundary 2410 of a cistern magna (CM). The boundary 2410 of the cistern magna may be detected as a discontinuous region, but may be approximated to a continuous curve shape between the cerebellum region 2110 and the skull region 2120.

Subsequently, in operation S2306, the image processor 110 measures a distance from an intersection point 2432 on a contour line 2430 of the cerebellum region 2110 to an intersection point 2412 on a boundary 2422 of a cistern magna region on a symmetrical line 2420 of a midsagittal plane to measure a size of a spinal fluid space of the CM. Here, the image processor 110 may determine the intersection point 2412 at which the symmetrical line 2420 intersects the boundary line 2422 of the cistern magna, the boundary line 2422 being closer to the cerebellum region 2110, and determine the intersection point 2432 at which the symmetrical line 2420 intersects the contour line 2430 of the cerebellum region 2110, the contour line 2430 being closer to the cistern magna. The image processor 110 may measure the distance between the intersection points 2412 and 2432 to measure the size of the spinal fluid space of the CM.

The symmetrical line 2420 of the midsagittal plane may be determined by approximating a line which passes through a symmetrical line region (2130 in FIG. 21) of a skull.

According to an exemplary embodiment, the image processor 110 may determine the symmetrical line 2420 of the midsagittal plane, approximate the “8”-shaped cerebellum region 2110 to two ellipses with respect to the symmetrical line 2420 of the midsagittal plane, and measure the size of the spinal fluid space of the CM.

According to another exemplary embodiment, the image processor 110 may approximate the cerebellum region 2110 to two ellipses with respect to the symmetrical line 2420 of the midsagittal plane, determine the symmetrical line 2420 of the midsagittal plane, and measure the size of the spinal fluid space.

A configuration for measuring the size of the spinal fluid space may be applied to a 2D brain image. For example, as described above, the image processor 110 may measure a size of the spinal fluid space in a 2D brain image representing the transcerebellar plane.

Moreover, according to an exemplary embodiment, when a brightness difference, a shape difference, or a size difference between the two ellipses approximating the cerebellum deviates from a reference range, the image processor 110 may again search for the sagittal plane to determine a direction of the sagittal plane.

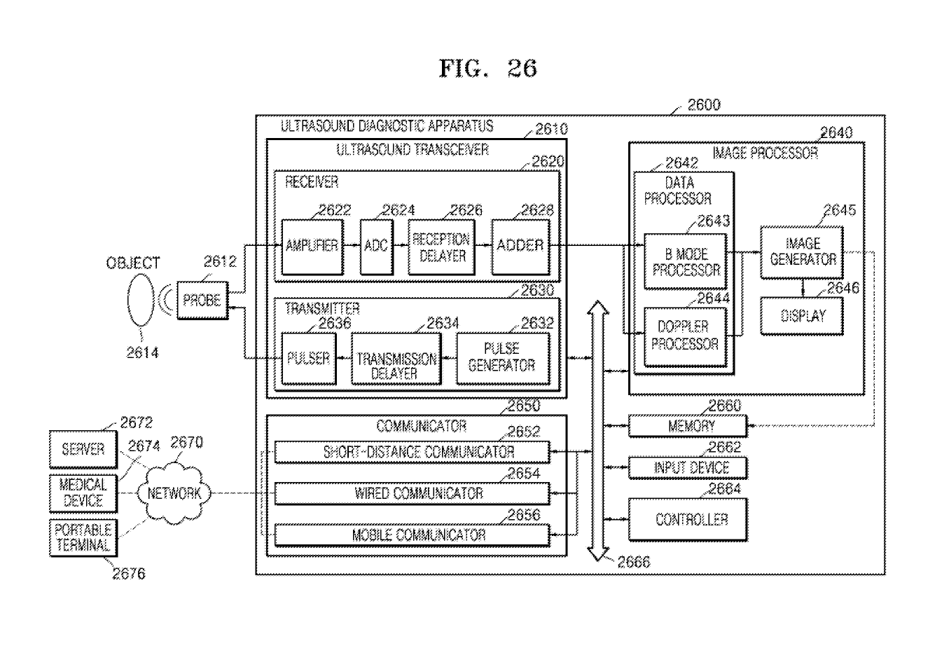

FIG. 25 is a flowchart illustrating an operation of detecting a POI, according to an exemplary embodiment.

In operation 52502, the image processor 110 detects a POI from a 3D brain image. Examples of the POI may include a sagittal plane, a transthalamic plane, a transventricular plane, and a transcerebellar plane.

Subsequently, in operation 52504, the image processor 110 detects a parameter from the POI. For example, the image processor 110 may measure a skull size in the sagittal plane, measure a ventricle size in the transthalamic plane, or measure a cerebellum size and a size of a spinal fluid in the transcerebellar plane.

Subsequently, the image processor 110 determines whether the parameter deviates from a reference range in operation 52506, and when the parameter deviates from the reference range, the image processor 110 re-detects the POI in operation 52508.

For example, when a change rate of the skull size deviates from a reference range, the image processor 110 may re-detect the transventricular plane, or re-detect the sagittal plane. Also, when a contrast of a boundary of a ventricle region and a contrast of a boundary of a CP region deviate from a reference range, the image processor 110 may re-detect the transventricular plane, or re-detect the sagittal plane.

For example, when a change rate of the cerebellum size or a change rate of the spinal fluid size deviates from a reference range, the image processor 110 may re-detect the transcerebellar plane, or re-detect the sagittal plane. Also, when the brightness difference, shape difference, or size difference between two ellipses approximating the cerebellum deviates from the reference range, the image processor 110 may again search for the sagittal plane to determine the direction of the sagittal plane.

FIG. 26 is a block diagram illustrating a configuration of an ultrasound diagnostic apparatus 2600 according to an exemplary embodiment. The image processing apparatus 100 according to an exemplary embodiment may be implemented as a type of the ultrasound diagnostic apparatus 2600.

Referring to FIG. 26, the ultrasound diagnostic apparatus 2600 according to an exemplary embodiment includes a probe 2612, an ultrasound transceiver 2610, an image processor 2640, a communicator 2650, a memory 2660, an input device 2662, and a controller 2664. The above-described elements may be connected to each other through a bus 2666.

The ultrasound diagnostic apparatus 2600 may be implemented as a portable diagnostic apparatus as well as a card type. Examples of the portable diagnostic apparatuses may include picture archiving and communication system (PACS) viewers, smartphones, laptop computers, personal digital assistants (PDAs), tablet personal computers (PCs), etc., but are not limited thereto.

The probe 2612 transmits ultrasound waves to an object 2614 based on a driving signal applied by the ultrasound transceiver 2610 and receives echo signals reflected by the object 2614. The probe 2612 includes a plurality of transducers, and the plurality of transducers oscillate based on electric signals transmitted thereto and generate acoustic energy, that is, ultrasound waves. Furthermore, the probe 2612 may be connected to a main body of the ultrasound diagnostic apparatus 2600 by a wire or wirelessly. According to exemplary embodiments, the ultrasound diagnostic apparatus 2600 may include a plurality of probes 2612.

The transceiver 2610 may include a receiver 2620 and a transmitter 2630. The transmitter 2630 supplies a driving signal to the probe 2612 and includes a pulse generator 2632, a transmission delayer 2634, and a pulser 2636. The pulse generator 2632 generates pulses for forming transmission ultrasound waves based on a predetermined pulse repetition frequency (PRF), and the transmission delayer 2634 applies a delay time for determining transmission directionality to the pulses. Pulses to which a delay time is applied correspond to a plurality of piezoelectric vibrators included in the probe 2612, respectively. The pulser 2636 applies a driving signal (or a driving pulse) to the probe 2612 as a timing corresponding to each pulse to which a delay time is applied.

The receiver 2620 generates ultrasound data by processing echo signals received from the probe 2612 and may include an amplifier 2622, an analog-to-digital converter (ADC) 2624, a reception delayer 2626, and an adder 2628. The amplifier 2622 amplifies echo signals in each channel, and the ADC 2624 analog-to-digital converts the amplified echo signals. The reception delayer 2626 applies delay times for determining reception directionality to the digital converted echo signals, and the adder 2628 generates ultrasound data by adding the echo signals processed by the reception delayer 2626. The receiver 2620 may omit the amplifier 2622 depending on an embodiment. That is, when a sensitivity of the probe 2612 is enhanced or the number of bits processed by the ADC 2624 increases, the amplifier 2622 may be omitted.

The image processor 2640 generates an ultrasound image by scan-converting ultrasound data generated by the ultrasound transceiver 2610 and displays the ultrasound image. An ultrasound image may include not only a grayscale ultrasound image obtained by scanning an object in an amplitude (A) mode, a brightness (B) mode, and/or a motion (M) mode, but also a blood flow Doppler image (also referred to as a color Doppler image) showing blood flow, a tissue Doppler image showing movement of tissues, and/or a spectral Doppler image showing moving speed of an object as a waveform.

A B mode processor 2643 extracts B mode components from ultrasound data and processes the B mode components. An image generator 2645 may generate an ultrasound image in which brightness is used to indicate signal intensities based on the extracted B mode components.

Similarly, a Doppler processor 2644 may extract Doppler components from ultrasound data, and the image generator 2645 may generate a Doppler image in which colors or waveforms are used to indicate movement of an object based on the extracted Doppler components.

The image generator 2645 according to an exemplary embodiment may generate a 2D ultrasound image via volume-rendering of volume data and may also generate an elasticity image which visualizes deformation of the object 2614 due to pressure. Furthermore, the image generator 2645 may display various additional information in an ultrasound image by using texts and graphics. The generated ultrasound image may be stored in the memory 2660.

The display 2646 displays the ultrasound image generated by the image generator 2645. The display 2646 may display various pieces of information processed by the ultrasound diagnostic apparatus 2600, in addition to the ultrasound image, on a screen through a graphics user interface (GUI). The ultrasound diagnostic apparatus 2600 may include two or more displays 2646 depending on an embodiment.

The communicator 2650 is connected to a network 2670 in a wired or wireless manner to communicate with an external device (e.g., a medical device 2674 or a portable terminal 2676) or a server 2672. The communicator 2650 may exchange data with a hospital server or a medical apparatus of a hospital which is connected to the communicator 2650 through a medical image information system (e.g., a PACS). Also, the communicator 2650 may perform data communication according to the digital imaging and communications in medicine (DICOM) standard.

The communicator 2650 may transmit and receive data, such as an ultrasound image, ultrasound data, Doppler data, etc. of the object 2614, associated with a diagnosis of the object 2614 over the network 2670, and may also transmit and receive a medical image captured by a medical apparatus such as a computed tomography (CT) apparatus, a magnetic resonance imaging (MRI) apparatus, or an X-ray apparatus. Furthermore, the communicator 2650 may receive information on a diagnosis history or a treatment schedule of a patient from a server, and use the received information in a diagnosis of the object 2614. In addition, the communicator 2650 may perform data communication with a portable terminal of a doctor or a patient, in addition to a server or a medical apparatus of a hospital.

The communicator 2650 may be connected to the network 2670 in a wired or wireless manner, and may exchange data with the server 2672, the medical apparatus 2674, or the portable terminal 2676. The communicator 2650 may include one or more elements that enable communication with an external device, and for example, include a short-distance communicator 2652, a wired communicator 2654, and a mobile communicator 2656.

The short-distance communicator 2652 performs short-distance communication within a certain distance. Short-distance communication technology, according to an exemplary embodiment, may include wireless local area network (LAN), Wi-Fi, Bluetooth, Zigbee, Wi-Fi direct (WFD), ultra wideband (UWB), infrared data association (IrDA), Bluetooth low energy (BLE), and near field communication (NFC), but not limited thereto.

The wired communicator 2654 performs communication using an electrical signal or an optical signal. Wired communication technology according to an exemplary embodiment may include a pair cable, a coaxial cable, an optical fiber cable, or an Ethernet cable.

The mobile communicator 2656 transmits and receives a radio frequency (RF) signal to and from a base station, an external terminal, and a server over a mobile communication network. Here, the RF signal may include various types of data based on transmission and reception of a voice call signal, a video call signal, or a text and/or multimedia message.

The memory 2660 stores various pieces of information processed by the ultrasound diagnostic apparatus 2600. For example, the memory 2660 may store medical data, such as input and/or output ultrasound data and ultrasound images, associated with a diagnosis of the object 2614, and may also store an algorithm or a program which is executed in the ultrasound diagnostic apparatus 2600.

The memory 2660 may be configured with various kinds of storage mediums such as a flash memory, a hard disk, an electrically erasable programmable read-only Memory (EEPROM), etc. Also, the ultrasound diagnostic apparatus 2600 may operate web storage or a cloud server which performs a storage function of the memory 2660 on a web.

The input device 2662 receives data, which is used to control the ultrasound diagnostic apparatus 2600, from a user. The input device 2662 may include hardware elements such as a keypad, a mouse, a touch pad, a trackball, a jog switch, but is not limited thereto. As another example, the input device 2662 may further include various sensors such as an electrocardiogram (ECG) device, a breath measurement sensor, a voice recognition sensor, a gesture recognition sensor, a fingerprint recognition sensor, an iris recognition sensor, a depth sensor, a distance sensor, etc.

The controller 2664 controls an overall operation of the ultrasound diagnostic apparatus 2600. That is, the controller 2664 may control operations between the probe 2612, the ultrasound transceiver 2610, the image processor 2640, the communicator 2650, the memory 2660, and the input device 2662, which are illustrated in FIG. 26.

Some or all of the probe 2612, the ultrasound transceiver 2610, the image processor 2640, the communicator 2650, the memory 2660, the input device 2662, and the controller 2664 may be operated by a software element, but are not limited thereto. Some of the above-described elements may be operated by a hardware element. Also, at least some of the ultrasound transceiver 2610, the image processor 2640, and the communicator 2650 may be included in the controller 2664, but are not limited to.

According to an exemplary embodiment, the image processor 110 of FIG. 1 may correspond to the image processor 2640 of FIG. 26.

According to an exemplary embodiment, the output unit 120 of FIG. 1 may correspond to at least one of the memory 2660, the display 2646, and the communicator 2650. When the output unit 120 is implemented as a type of the memory 2660, an image file storing a medical image generated by the image processor 110 may be stored in the memory 2600. When the output unit 120 is implemented as a type of the display 2646, the medical image generated by the image processor 110 may be displayed on the display 2646. When the output unit 120 is implemented as a type of the communicator 2650, the medical image generated by the image processor 110 may be transmitted to the server 2672, the medical apparatus 2674, and/or the portable terminal 2676.

According to another exemplary embodiment, the medical image processing apparatus 100 may be implemented as a type of a CT diagnostic apparatus or an MRI diagnostic apparatus.

As described above, according to the one or more of the above exemplary embodiments, a POI desired by a user is automatically determined in a 3D brain image.

According to the one or more exemplary embodiments, parameters of a brain image are automatically measured.

The medical image processing method according to exemplary embodiments may be embodied as an algorithm or a computer program and may be stored on a computer-readable recording medium as computer readable codes or program commands executable by a processor. Examples of the computer-readable recording medium include magnetic storage media (e.g., read-only memories (ROMs), floppy disks, hard disks, etc.), optical recording media (e.g., compact disk (CD)-ROMs, or digital versatile disks (DVDs)), and the like. The computer-readable recording medium may also be distributed over network-coupled computer systems so that the computer readable code is stored and executed in a distributed fashion. The recoding medium may be read by a computer, stored in a memory, and executed by the processor. Also, when the recording medium is connected to the medical image processing apparatus 100, the recording medium may be implemented in order for the medical image processing apparatus 100 to perform the medical image processing method according to exemplary embodiments.

The foregoing exemplary embodiments and advantages are merely exemplary and are not to be construed as limiting. The present teaching can be readily applied to other types of apparatuses. The description of the exemplary embodiments is intended to be illustrative, and not to limit the scope of the claims, and many alternatives, modifications, and variations will be apparent to those skilled in the art

What is claimed is:

1. A medical image processing apparatus comprising:
an image processor configured to detect an anatomical organ from a three-dimensional (3D) brain image, and determine a plane-of-interest (POI) from the 3D brain image, based on the detected anatomical organ; and
an output unit configured to output an image of the POI.

2. The medical image processing apparatus of claim 1, wherein,
the POI is a sagittal plane, and
the image processor is configured to detect an elliptical shape of a maximum size and detect a cavum septi pellucidi (CSP) and a cerebellum, while moving a two-dimensional (2D) plane in the 3D brain image, and
the image processor is configured to determine the 2D plane, in which the elliptical shape of the maximum size is detected and the CSP and the cerebellum are detected, as the sagittal plane.

3. The medical image processing apparatus of claim 1, wherein,
the POI is a transthalamic plane,
the image processor is configured to detect a CSP from a sagittal plane of the 3D brain image, set a candidate plane of the transthalamic plane, the candidate plane being vertical to the sagittal plane based on the detected CSP, and detect a skull and a region forming a trident shape from the candidate plane of the transthalamic plane, and
the image processor is configured to determine a candidate plane of the transthalamic plane, in which a size of the skull has a maximum value and the region forming the trident shape is detected, as the transthalamic plane.

4. The medical image processing apparatus of claim 3, wherein the image processor is configured to set, as candidate planes of the transthalamic plane, a CSP plane which is vertical to the sagittal plane and includes a straight line, the strait line contacting the CSP and parallel to an elongated direction of the CSP, and a plane parallel to the CSP plane.

5. The medical image processing apparatus of claim 3, wherein, when a change rate of the size of the skull deviates from a reference range, the image processor is configured to re-detect the sagittal plane.

6. The medical image processing apparatus of claim 3, wherein the image processor is configured to measure the size of the skull in the transthalamic plane.

7. The medical image processing apparatus of claim 1, wherein
the POI is a transventricular plane,
the image processor is configured to detect a CSP from a sagittal plane of the 3D brain image, seta candidate plane of the transventricular plane, the candidate plane being vertical to the sagittal plane, based on the detected CSP, and detect a choroid plexus and a ventricle from the candidate plane of the transventricular plane, and
the image processor is configured to determine the transventricular plane according to a result of the detection of the choroid plexus and the ventricle from the candidate plane of the transventricular plane.

8. The medical image processing apparatus of claim 7, wherein the image processor is configured to set, as candidate planes of the transventricular plane, a CSP plane which is vertical to the sagittal plane and includes a straight line, the straight line contacting the CSP and parallel to an elongated direction of the CSP, and a plane parallel to the CSP plane.

9. The medical image processing apparatus of claim 7, wherein the image processor is configured to determine the transventricular plane, based on at least one of a contrast of a boundary of a region corresponding to the choroid plexus, a size of the choroid plexus, and a contrast of a boundary of a region corresponding to the ventricle.

10. The medical image processing apparatus of claim 7, wherein the image processor is configured to detect a center of a choroid plexus region and a center of a ventricle region, determine a central line bisecting a line, the line connecting the center of the choroid plexus region and the center of the ventricle region, determine a first straight line approximating an upper boundary of the choroid plexus region and the ventricle region and a second straight line approximating a lower boundary of the choroid plexus region and the ventricle region, and determine, as a size of the ventricle, a distance between two points at which the first and second straight lines intersect the central line, respectively.

11. The medical image processing apparatus of claim 10, wherein the image processor is configured to determine the first and second straight lines based on an angle between the central line and the first straight line and an angle between the central line and the second straight line.

12. The medical image processing apparatus of claim 1, wherein,
the POI is a transcerebellar plane,
the image processor is configured to detect a CSP and a cerebellum from a sagittal plane, set a candidate plane of the transcerebellar plane, the candidate plane being vertical to the sagittal plane and including a straight line connecting the CSP and the cerebellum, and detect the cerebellum from the candidate plane of the transcerebellar plane, and
the image processor is configured to determine the transcerebellar plane according to a result of the detection of the cerebellum from the candidate plane of the transcerebellar plane.

13. The medical image processing apparatus of claim 12, wherein the image processor is configured to detect a skull from the sagittal plane, determine a symmetrical line of the skull, and detect, as the cerebellum, a region having substantially an “8” shape and vertically contacting the symmetrical line.

14. The medical image processing apparatus of claim 13, wherein the image processor is configured to measure a length of a line, which connects a highest point and a lowest point of the detected region in a vertical direction, and determine the measured length as a size of the cerebellum.

15. The medical image processing apparatus of claim 13, wherein the image processor is configured to detect a cistern magna from the transcerebellar plane, and measure, as a size of a spinal fluid space, a distance between a point, at which two circles or ellipses of the “8” shape of the detected region contact each other, and the cistern magna.

16. The medical image processing apparatus of claim 13, wherein, when at least one of a brightness difference, a shape difference, and a size difference between two circles or ellipses of the “8” shape of the detected region is equal to or greater than a reference range, the image processor is configured to re-detect the sagittal plane.

17. The medical image processing apparatus of claim 1, wherein the image processor is configured to automatically detect a certain parameter from the POI, and in response to the detected parameter being deviated from a reference range, the image processor is configured to re-determine the POI.

18. The medical image processing apparatus of claim 17, wherein the certain parameter is determined according to a type of the POI.

19. A medical image processing method comprising:
detecting an anatomical organ from a three-dimensional (3D) brain image;
determining a plane-of-interest (POI) from the 3D brain image, based on the detected anatomical organ; and
outputting an image of the POI.

20. The medical image processing method of claim 19, wherein,
the POI is a sagittal plane,
the detecting the anatomical organ comprises:
detecting an elliptical shape of a maximum size and detecting a cavum septi pellucidi (CSP) and a cerebellum, while moving a two-dimensional (2D) plane in the 3D brain image, and the determining the POI comprises determining the 2D plane, in which the elliptical shape of the maximum size is detected and the CSP and a cerebellum are detected, as the sagittal plane.

21. The medical image processing method of claim 19, wherein,
the POI is a transthalamic plane,
the detecting the anatomical organ comprises:
detecting a CSP from a sagittal plane of the 3D brain image;
setting a candidate plane of the transthalamic plane, the candidate plane being vertical to the sagittal plane based on the detected CSP; and
detecting a skull and a region forming a trident shape from the candidate plane of the transthalamic plane, and
the determining the POI comprises determining a candidate plane of the transthalamic plane, in which a size of the skull has a maximum value and the region forming the trident shape is detected, as the transthalamic plane.

22. The medical image processing method of claim 21, wherein the setting the candidate plane of the transthalamic plane comprises setting, as candidate planes of the transthalamic plane, a CSP plane which is vertical to the sagittal plane and includes a straight line, the straight line contacting the CSP and parallel to an elongated direction of the CSP, and a plane parallel to the CSP plane.

23. The medical image processing method of claim 21, further comprising, when a change rate of the size of the skull deviates from a reference range, re-detecting the sagittal plane.

24. The medical image processing method of claim 21, further comprising measuring the size of the skull in the transthalamic plane.

25. The medical image processing method of claim 19, wherein
the POI is a transventricular plane,
the detecting the anatomical organ comprises:
detecting a CSP from a sagittal plane of the 3D brain image;
setting a candidate plane of the transventricular plane, the candidate plane being vertical to the sagittal plane, based on the detected CSP; and
detecting a choroid plexus and a ventricle from the candidate plane of the transventricular plane, and
the determining the POI comprises determining the transventricular plane according to a result of the detecting the choroid plexus and the ventricle from the candidate plane of the transventricular plane.

26. The medical image processing method of claim 25, wherein the setting the candidate plane of the transventricular plane comprises setting, as candidate planes of the transthalamic plane, a CSP plane which is vertical to the sagittal plane and includes a straight line, the straight line contacting the CSP and parallel to an elongated direction of the CSP, and a plane parallel to the CSP plane.

27. The medical image processing method of claim 25, wherein the determining the transventricular plane comprises determining the transventricular plane, based on at least one of a contrast of a boundary of a region corresponding to the choroid plexus, a size of the choroid plexus, and a contrast of a boundary of a region corresponding to the ventricle.

28. The medical image processing method of claim 25, further comprising:
detecting a center of a choroid plexus region and a center of a ventricle region;
determining a central line bisecting a line, the line connecting the center of the choroid plexus region and the center of the ventricle region;
determining a first straight line approximating an upper boundary of the choroid plexus region and the ventricle region and a second straight line approximating a lower boundary of the choroid plexus region and the ventricle region; and
determining, as a size of the ventricle size, a distance between two points at which the first and second straight lines intersect the central line, respectively.

29. The medical image processing method of claim 28, wherein the determining the distance as the ventricle size comprises determining the first and second straight lines based on an angle between the central line and the first straight line and an angle between the central line and the second straight line.

30. The medical image processing method of claim 19, wherein,
the POI is a transcerebellar plane,
the detecting the anatomical organ comprises:
detecting a CSP and a cerebellum from a sagittal plane;
setting a candidate plane of the transcerebellar plane, the candidate plane being vertical to the sagittal plane and including a straight line connecting the CSP and the cerebellum; and
detecting the cerebellum from the candidate plane of the transcerebellar plane, and
the determining the POI comprises determining the transcerebellar plane according to a result of the detecting the cerebellum from the candidate plane of the transcerebellar plane.

31. The medical image processing method of claim 30, wherein the detecting the CSP and the cerebellum comprises:
detecting a skull from the sagittal plane, and determining a symmetrical line of the skull; and
detecting, as the cerebellum, a region having substantially an “8” shape and vertically contacting the symmetrical line.

32. The medical image processing method of claim 31, further comprising measuring a length of a line, which connects a highest point and a lowest point of the detected region in a vertical direction, and determining the measured length as a size of the cerebellum.

33. The medical image processing method of claim 31, further comprising:
detecting a cistern magna from the transcerebellar plane; and
measuring, as a size of a spinal fluid space, a distance between a point, at which two circles or ellipses of the “8” shape of the detected region contact each other, and the cistern magna.

34. The medical image processing method of claim 31, further comprising re-detecting the sagittal plane when at least one of a brightness difference, a shape difference, and a size difference between the two circles or ellipses of the “8’ shape of the detected region is equal to or greater than a reference range.

35. The medical image processing method of claim 17, wherein the determining the POI comprises automatically detecting a certain parameter from the POI, and in response to the detected parameter being deviated from a reference range, re-determining the POI.

36. The medical image processing method of claim 35, wherein the certain parameter is determined according to a type of the POI.

37. A non-transitory computer-readable storage medium storing a program which, when executed by a computer, performs a medical image processing method comprising:
detecting an anatomical organ from a three-dimensional (3D) brain image;
determining a plane-of-interest (POI) from the 3D brain image, based on the detected anatomical organ; and
outputting an image of the POI.
